(19) United States
(12) Patent Application Publication
(10) Pub. No.: US 2024/0081975 A1
(43) Pub. Date: Mar. 14, 2024

(54) TORIC INTRAOCULAR LENS ALIGNMENT GUIDE

(71) Applicant: Alcon Inc., Fribourg (CH)

(72) Inventors: Vignesh Suresh, Arlington, TX (US); Lu Yin, Keller, TX (US); Ramesh Sarangapani, Coppell, TX (US); Tobias Jura Rapoport, Berlin (DE)

(21) Appl. No.: 18/464,050

(22) Filed: Sep. 8, 2023

Related U.S. Application Data
(60) Provisional application No. 63/406,084, filed on Sep. 13, 2022.

(51) Int. Cl. A61F 2/16; A61B 34/10

(52) U.S. Cl. CPC A61F 2/16; A61B 34/10; A61B 2034/105; A61B 2034/107

(57) ABSTRACT

Particular embodiments disclosed herein provide an alignment guide for aligning a toric IOL during surgery. An image with a reference axis is obtained, such as from a digital microscope, and processed, such as using an autoencoder, to label alignment marks on the IOL and possibly other features of the IOL. The label is processed, such as using a logistic regression model, to estimate an IOL axis of the IOL intersecting the alignment marks. An output image is generated from the image that has superimposed thereon guides to a surgeon, such as a line representing the IOL axis, a rotation direction indicator, and a number or other representation of a difference between the reference axis and the IOL axis. Tracking of features of the IOL may be performed across multiple images to predict the location of features not represented in a particular image.

### TECHNICAL FIELD

The present disclosure relates generally to methods for the treatment of cataracts and, more particularly, to treatments including the use of intraocular lenses (IOL).

### BACKGROUND

Light received by the human eye, passes through the transparent cornea covering the iris and pupil of the eye. The light is transmitted through the pupil and is focused by a crystalline lens positioned behind the pupil in a structure called the capsular bag. The light is focused by the lens onto the retina, which includes rods and cones capable of generating nerve impulses in response to the light.

Through age or disease, the crystalline lens may become cloudy, a condition known as a cataract. Cataracts are a readily treated by removing the crystalline lens and inserting an artificial lens, known as an intraocular lens (IOL). The IOL may be fabricated to additionally correct for aberrations of the patient's eye, such as astigmatism. Inasmuch as astigmatism is the result of asymmetry of the eye, the IOL must be aligned with the asymmetry of the eye in order to compensate for it. The IOL is therefore provided with markers, such as rows of dots at the perimeter of the IOL, which define an axis that may be used to align the IOL. The IOL may be implemented as a toric IOL, which includes spring-like arms, known as haptics, which hold the IOL in place within the capsular bag. In prior approaches, an imaging device, such as a digital marker microscope (DMM), is used to view the patient's eye during surgery. The image output by the imaging device has a reference axis superimposed thereon that corresponds to the desired orientation of the axis of the IOL.

Inasmuch as precise alignment of the IOL axis with the reference is desired, approaches for facilitating this alignment would greatly improve patient outcomes.

### BRIEF SUMMARY

The present disclosure relates generally to a system providing an alignment guide for positioning a toric IOL in a patient's eye.

Particular embodiments disclosed herein provide a method and corresponding apparatus for providing alignment guidance during ocular surgery. The method includes receiving, by a computing device, from an imaging device, an input image of a patient's eye having a toric intraocular lens (IOL) within the patient's eye. The computing device obtains a reference axis for the patient's eye, the reference axis indicating a desired orientation of a toric IOL axis of the toric IOL. The input image is processed to obtain a feature label indicating locations of features of the toric IOL represented in the input image, the features including any of: alignment dots defined on the toric IOL, a perimeter of the toric IOL, and portions of haptics of the toric IOL. The feature label is processed by the computing device to determine an orientation of the toric IOL axis. The method then includes calculating an angle difference between the toric IOL axis and the reference axis. The computing device then generates and outputs an output image including at least one indicator corresponding to the angle difference.

The following description and the related drawings set forth in detail certain illustrative features of one or more embodiments.

### BRIEF DESCRIPTION OF THE DRAWINGS

The appended figures depict certain aspects of the one or more embodiments and are therefore not to be considered limiting of the scope of this disclosure.

FIG. 1 illustrates anatomy of the human eye.

FIG. 2 illustrates a toric IOL.

FIG. 3A illustrates an alignment guide system for positioning a toric IOL, in accordance with certain embodiments.

FIG. 3B illustrates a post processor for the alignment guide system, in accordance with certain embodiments.

FIG. 4A illustrates a method for providing an alignment guide for positioning a toric IOL, in accordance with certain embodiments.

FIG. 4B is a method for performing post processing, in accordance with certain embodiments.

FIG. 5A to FIG. 5F illustrate the provision of an alignment guide on images of a patient's eye while undergoing placement of a toric IOL, in accordance with certain embodiments.

To facilitate understanding, identical reference numerals have been used, where possible, to designate identical elements that are common to the drawings. It is contemplated that elements and features of one embodiment may be beneficially incorporated in other embodiments without further recitation.

### DETAILED DESCRIPTION

Particular embodiments of the present disclosure provide an alignment guide for positioning a toric IOL in a patient's eye.

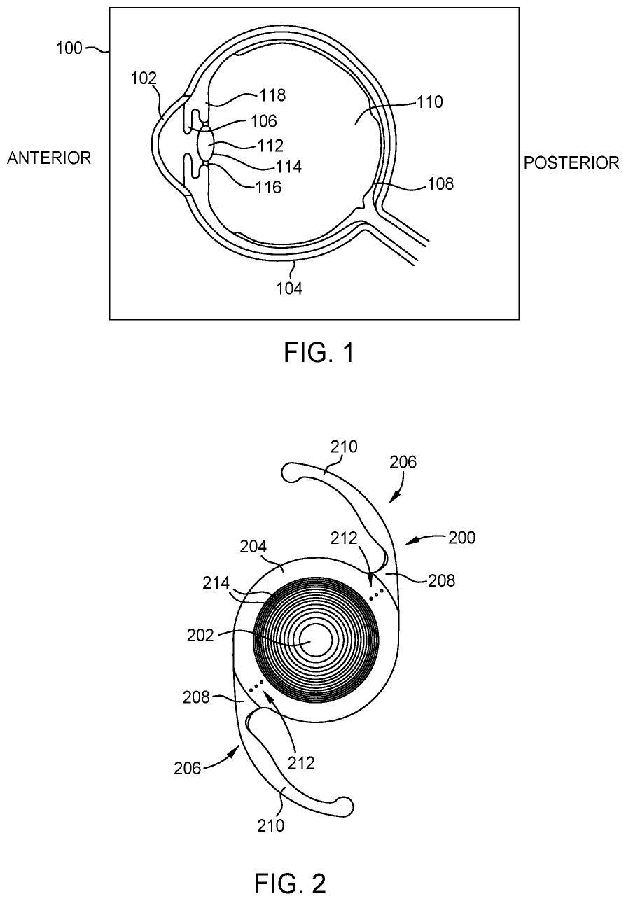

FIG. 1 is a diagram illustrating parts of the human eye 100 that may be understood with respect to the anterior side, through which light enters the eye, and the posterior side opposite the anterior side. At the anterior side of the eye 100, a thin transparent layer known as the cornea 102 is linked to the sclera 104, which forms the generally spherical wall of the eye 100. The cornea 102 and sclera 104 are connected by a ring called the limbus. The iris 106, the color of the eye, and an opening defined by it, the pupil, are positioned behind the cornea and are visible due to the cornea's 102 transparency. The retina 108 is formed on an interior surface of the sclera 104 opposite the cornea 102 and iris 16. The volume defined by the sclera 104 is occupied by the transparent jelly of the vitreous body 110.

The crystalline lens 112 is a transparent, biconvex structure in the eye that, along with the cornea 102, helps to refract light to be focused on the retina 108. The lens 112, by changing its shape, functions to change the focal distance of the eye so that it can focus on objects at various distances, thus allowing a sharp real image of the object of interest to be formed on the retina 108. This adjustment of the lens 112 is known as accommodation, and is similar to the focusing of a photographic camera via movement of its lenses.

The lens 112 is positioned behind the iris 106 in a capsular bag 114. The capsular bag 114 is attached at its perimeter to the suspensory ciliary ligament 116. The ciliary ligament 116 attaches the capsular bag 114 to the ciliary body 118. The ciliary body 118 is a ring-shaped muscle that attaches the ciliary ligament 116 to the sclera 104 and which can contract or relax in order to change the shape of the lens 112.

Various diseases and disorders of the lens 112 may be treated with an IOL. By way of example, not necessarily limitation, an IOL according to embodiments of the present disclosure may be used to treat cataracts, large optical errors in myopic (near-sighted), hyperopic (far-sighted), and astigmatic eyes, ectopia lentis, aphakia, pseudophakia, and nuclear sclerosis. However, for purposes of description, the IOL embodiments of the present disclosure are described with reference to cataracts, which often occurs in the elderly population.

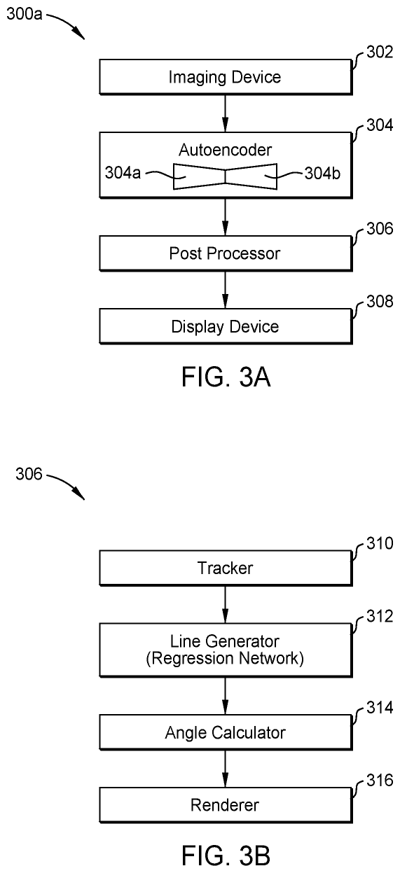

FIG. 2 illustrates an example toric IOL 200. The toric IOL 200 includes a lens portion 202 that focuses light passing through the iris 106 onto the retina 108. The lens portion 202 may be surrounded by a peripheral ring 204 that is not used to focus light. Two or more haptics 206 may secure to the peripheral ring 204. Each haptic 206 may include a base 208 secured to the peripheral ring 204 and extending outwardly therefrom. A spring arm 210 secures to the base and extends both outwardly from the peripheral ring 204 and circumferentially around the peripheral ring 204. In use, the spring arms 210 push outwardly against the capsular bag 114 and hold the toric IOL 200 in a desired position.

Marks 212 may be formed on the peripheral ring 204. The marks 212 facilitate alignment of the IOL with the eye 100 of the patient. The marks 212 in the illustrated toric IOL 200 include two sets of dots (e.g., depressions or bumps), such as circular dots, formed on the peripheral ring 204 opposite one another. For example, each set may include, two, three, or more dots. The dots of each set may be collinear with one another and be collinear with the dots of the other set. The line passing through the dots of one or both sets (hereinafter “the IOL axis”) may also intersect and be perpendicular to the optical axis of the lens portion 202. In the illustrated toric IOL 200, the IOL axis also intersects the bases 208 of the haptics 206. Some toric IOLs 200 are multi-focal. The lens portion 202 may include rings 214 that define the boundary between regions of the lens portion 202 with different focal lengths. As discussed below, the marks 212 may be detected using a machine learning model and used to determine the IOL axis. Accordingly, the marks 212 need not be intersected by the IOL axis and may include any arbitrary pattern that is visible in an image of the IOL 200. The machine learning model may be trained to identify the marks 212 of whatever shape, arrangement, and number. As also discussed below, features other than the marks 212 may be used to determine the orientation of the IOL 200, such as the bases 208 of the haptics, the perimeter of the IOL 200. The geometrical relationships between any two or more features may be used to determine the orientation of the IOL 200 using the machine learning model as outlined below.

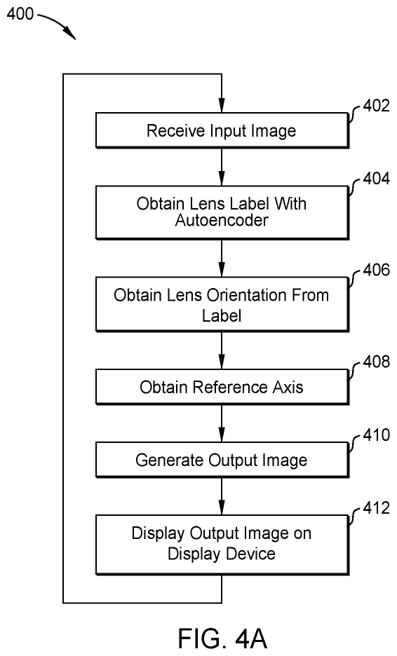

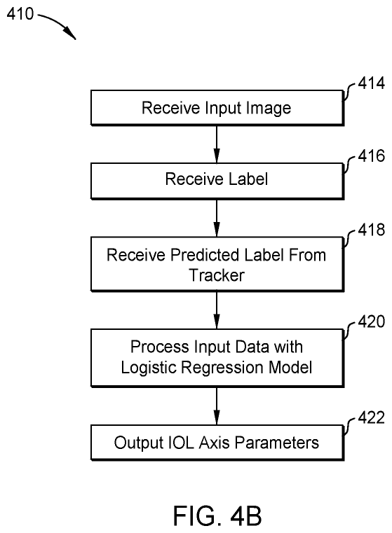

FIG. 3A illustrates an alignment guide system 300a for positioning a toric IOL 200 in the eye 100 of a patient. The alignment guide system 300a may include an imaging device 302 for capturing an image of the patient's eye 100 during implantation and alignment of the toric IOL 200. The imaging device 302 may be embodied as a digital three-dimensional microscope, such as the ALCON NGENUITY 1.5 (a digital three-dimensional digital marker microscope (DMM) with integrated image guidance) or ZEISS ARTEVO, or as an analog microscope with image guidance, such as the ALCON VERION DIGITAL MARKER or ZEISS CALISTO. As known in the art, an imaging device 302 may be programmed with a treatment plan including pre-operative images of the patient's eye 100, which may include images of the sclera 104, iris 106 and possibly a portion of the retina 108. The imaging device 302 may further be configured to define a reference axis with respect to one or more images of the patient's eye 100. The imaging device 302 may be programmed to perform registration of the patient's eye 100. Registration may be performed by capturing an image of the patient's eye 100 and matching ocular anatomy, such as the unique patterns of blood vessels on the sclera 104 and/or retina 108 and/or the unique patterns of the limbus and/or iris 106 represented in the image, to representations of corresponding ocular anatomy in the one or more pre-operative images to determine the orientation of the eye 100. The imaging device 302 may then determine a transformation relating the orientation of the eye 100 in the image to the orientation of the eye in a reference image included in the treatment plan. The reference axis of the treatment plan may then be rotated and/or translated according to the transformation. References herein to the reference axis superimposed on an image of the patient's eye shall be understood as referring to the reference axis resulting from the transformation. Registration and corresponding transformation of the reference axis may be performed repeatedly for images (e.g., frames of video data) captured throughout a surgery to account for movement of the patient's eye relative to the imaging device 302.

The components of the system 300a in addition to the imaging device may be implemented using the computing capabilities of the imaging device 302 embodied as a digital microscope. Alternatively, the additional components of the system 300a may be implemented by a separate computing device that receives images labeled with the reference axis from the imaging device 302. In still other implementations, the separate computing device may receive no more than images from the imaging device 302 and perform registration with respect to the one or more pre-operative images as described above to obtain the reference axis.

The system 300a may include an autoencoder 304. The autoencoder 304 receives images output by the imaging device 302, which may be marked with the reference axis and identifies features of the IOL 200 represented in the image. These features may include some or all of the marks 212, rings 214, the bases 208 of the haptics, and a perimeter of the peripheral ring 204. The autoencoder 304 may label one or more of these features. The label may be in the form of one or more pixel masks in which non-zero pixels correspond to the pixels of the image found by the autoencoder 304 to represent one or more features or types of features. Separate pixel masks may be generated by the autoencoder 304 for each feature or type of feature, such as a mask for the marks 212, a mask for the rings 214, a mask for the bases 208 of the haptics, and a mask for the perimeter of the peripheral ring 204. The autoencoder 304 may generate multiple masks with one or more of the masks labeling multiple features or types of features. Alternatively, a single mask may mark pixels corresponding to any of the features.

The autoencoder 304 may include an encoder 304a and a decoder 304b such that the image is input to the encoder 304a and the output of the encoder 304a is input to the decoder 304b. The output of the decoder 304b may include the one or more pixel masks described above. The autoencoder 304 may be implemented using convolution neural networks (CNN), deep neural network (DNN), or other types of neural network. The autoencoder 304 may be replaced with any machine learning model known in the art that has been trained to perform the tasks ascribed herein to the autoencoder 304.

Training data for the autoencoder 304 may include an image of a patient's eye 100 as an input and, as a desired output, one or more pixel masks labeling the features of a toric IOL 200 positioned within the patient's eye 100. For example, during an implantation procedure, a video feed from a DMM may be captured and a plurality of frames from the video feed may be labeled by a human (e.g., a human-generated pixel mask marking pixels corresponding to features represented by the pixel mask) such that each frame and its corresponding label becomes a training data entry. Such training data entries from multiple patients may then be used to train the autoencoder 304 by processing the image of each training data entry with the autoencoder 304, receiving an output from the autoencoder 304, comparing the output of the autoencoder to the one or more pixel masks of the training data entry (e.g., evaluating a loss function), and updating parameters of the autoencoder 304 according to the comparison.

The autoencoder 304 may include, or be used in combination with, an attention mechanism that specifies one or more areas of an input image that contain or are likely to container features (marks 212, bases 208 of haptics 206, perimeter of the IOL 200, etc.). The attention mechanism may generate bounding boxes that contain or are likely to contain features. The attention mechanism may be another machine learning model, such as another autoencoder, or one or more layers of the autoencoder 304. The attention mechanism may be trained with training data entries that each include an image as an input and, as a desired output, one or more bounding boxes obtained from a human or computerized labeler, the one or more bounding boxes each labeling one or more features. A training algorithm may then process each training data entry by processing the input image using the attention mechanism to obtain one or more estimated bounding boxes. The one or more estimated bounding boxes may then be compared to the one or more bounding boxes of the training data entry. The training algorithm may then update parameters of the attention mechanism according to similarity of the one or more estimated bounding boxes to the one or more bounding boxes of the training data entry. The output of the attention mechanism may be an output image that is the input image having the pixels contained within one or more bounding boxes highlighted. The desired features are identified within the region highlighted by the bounding box by the subsequent layers of the autoencoder 304

The label output by the autoencoder 304 may be input to a post processor 306. The post processor 306 may determine the IOL axis from the label and determine an angular difference between the IOL axis and the reference axis. The post processor 306 may generate an output image based on the image received from the imaging device 302 that has information superimposed thereon, such as lines representing the reference axis and the IOL axis and arrows, text, or other information indicating an amount of rotation needed to align the IOL axis with the reference axis.

The output image generated by the post processor 306 may then be output to a display device 308, which may be a screen incorporated into the imaging device 302 and viewable through an eye piece of the imaging device 302. The display device 308 may be implemented as a monitor in a room in which the implantation of the toric IOL 200 is being performed, a heads-up display worn by a surgeon performing the implantation, or another display device.

FIG. 3B illustrates an example implementation of a post processor 306. The post processor 306 may include a tracker 310. The output of the autoencoder 304 may be a series of labels for each frame of a video feed from the imaging device 302. The tracker 310 may receive these labels, and possibly the frames of the video feed, and track movement of features represented by the labels from one frame to the next. The tracker 310 may therefore predict the location of a feature for frames in which the feature is not labeled due to (a) being obscured by a surgical instrument or a portion of the eye 100 (e.g., the iris 106) or (b) not being successfully identified by the autoencoder 304. The tracker 310 may be implemented as a Markov chain, flow detector, Kalman filter, or any other tracking algorithm known in the art.

The output of the tracker 310 for a frame of the video feed may be a label having the same form as the label received from the autoencoder 304. For example, the output of the tracker 310 may be a pixel mask (“tracker mask”) corresponding to each pixel mask output by the autoencoder 304 (“original mask”). The tracker mask may include new non-zero pixels at pixel positions that are zero in the original mask, the new non-zero pixels indicating a predicted location of a feature or portion of a feature that was not labeled in the original mask. The tracker 310 may also perform a degree of noise cancellation or smoothing that may cause pixel positions in the original mask and tracker mask to have different values (zero flipped to non-zero or non-zero flipped to zero).

The label as output by the tracker 310, or as output by the autoencoder 304 where a tracker 310 is not used, (“the label”) may be input to a line generator 312. The line generator 312 may be any machine vision algorithm trained or programmed to fit a line to labels of the marks 212. In some implementations, the line generator 312 is a machine learning model, such as a neural network implementing a logistic regression model, trained to generate line parameters (e.g., slope and x or y intercept) based on the label and possibly the image from which the label was generated (“the image”). As described above, the label itself may include one or more pixel masks.

The label may include more information than is necessary to define the IOL axis. Only two points are required to define a line, yet there may be 4, 6, or more dots included in the marks 212 and the bases 208 of the haptics may also be represented in the image. The machine learning model of the line generator 312 may advantageously use some or all of this information to accurately define a line representing the IOL axis. Training data entries for the machine learning model may include a label and possibly an image corresponding to the label as an input and, as a desired output, human-generated parameters (slope and x or y intercept) describing the IOL axis of a toric IOL represented in the image. For each training data entry, the input may be processed using the machine learning model to obtain estimated line parameters. The estimated line parameters may be compared to the parameters of the training data entry (e.g., a loss function may be evaluated) and parameters of the machine learning model may be updated according to the comparison. During utilization, the label from the autoencoder 304, and possibly the image from which the label was generated, may be processed using the machine learning model to obtain line parameters estimating the slope and location of the IOL axis.

The line parameters describing the IOL axis may be processed using an angle calculator 314. The angle calculator 314 compares the line parameters describing the IOL axis to parameters describing the reference axis and computes a difference in angle. For example, for two lines y1=m1*x+b1 and y2=m2*x+b1, the difference in angle may be calculated as ATAN((m1−m2)/(1+m1*m2)). The angle may be adjusted (e.g., subtracted from 180 degrees or Pi radians) to obtain a difference in angle to present to the surgeon. The toric IOL 200 may be safely rotatable in only one direction in some implementations, such as clockwise for the illustrated toric IOL 200. Any approach for calculating the angle difference between two lines may be used.

The difference in angle, and possibly the label, and the image may be processed using a renderer 316. The renderer 316 may superimpose some or all of the following on the image to obtain an output image:
A representation of the reference axis;
A representation of the IOL axis;
A direction indictor (e.g., arrow describing a direction of rotation to move the IOL axis to align with the reference axis)
A numerical and/or graphical representation of the difference in angle between the reference axis and the IOL axis. For example, a number indicating a number of degrees to rotate the toric IOL 200 to eliminate the difference in angle may be illustrated. In some embodiments colors are used: red representing a first range of differences in angle, yellow representing a second range of differences in angle below the first range, and green indicating a third range of differences in angle below the second range.

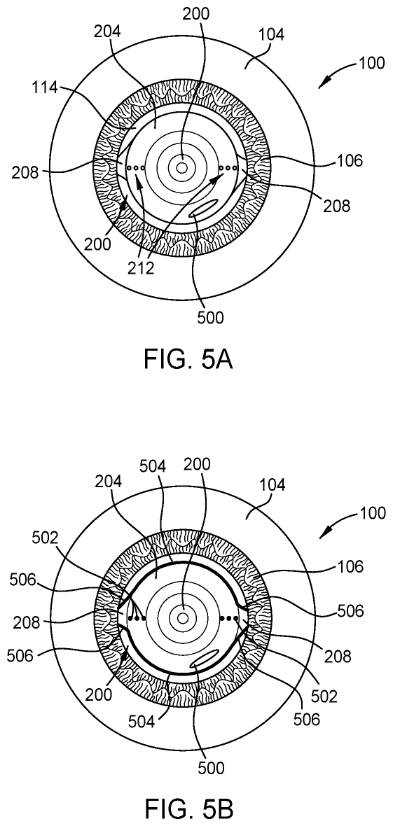

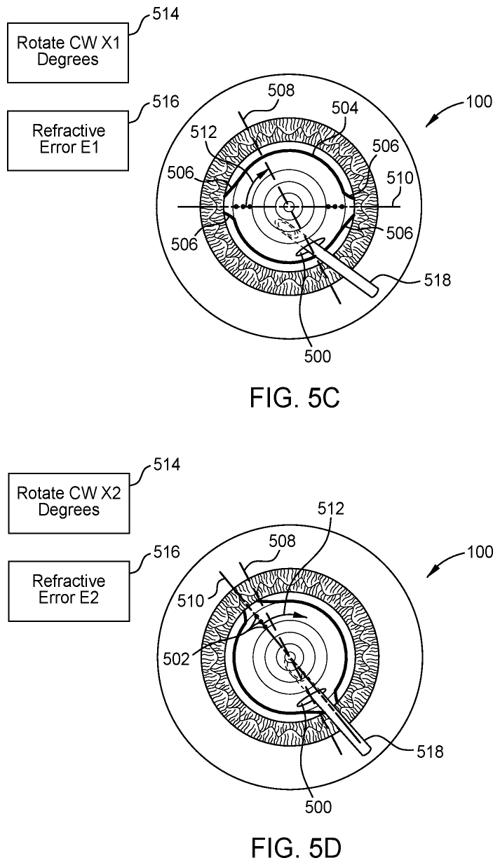

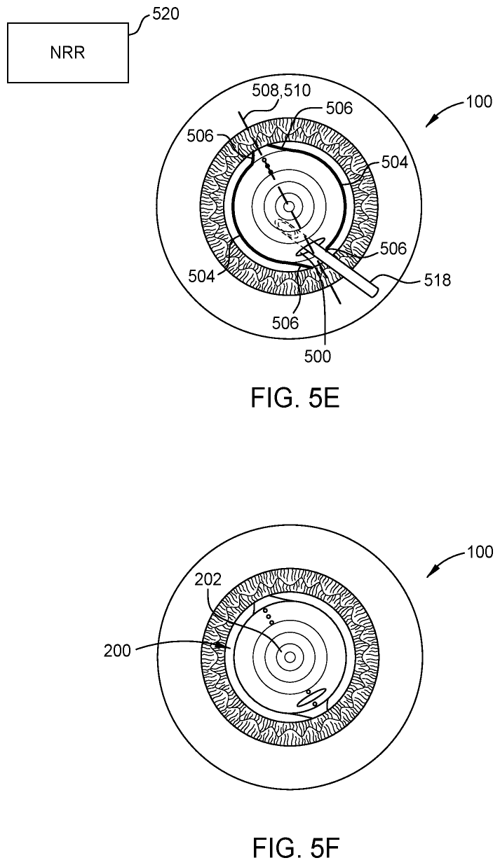

FIG. 4A and FIG. 4B illustrate a method 400 for providing an alignment guide for positioning a toric IOL 200, in accordance with certain embodiments. The method 400 is described with reference to the diagrams of FIGS. 5A to 5F. The method 400 may be executed by a computing device incorporated within the imaging device 302 or a different computing device coupled to the imaging device 302 and receiving images (e.g., a video feed) from the imaging device 302. Such a computing device includes one or more processing devices (e.g., central processing units (CPU)) and one or more memory devices coupled to the one or more processing devices, the one or more memory devices storing executable code including instructions that, when executed, causes the one or more processing devices to perform the method 400.

The method 400 may commence following implantation of the toric IOL 200 in the capsular bag 114. Steps performed in preparation for implantation of the toric IOL 200 and the initial placement of the toric IOL 200 in the capsular bag 114 may be performed according to any approach known in the art.

The method 400 may include receiving, at step 402, an input image from the imaging device 302. In the description of FIG. 4A and FIG. 4B and FIGS. 5A to 5F, reference to an item of anatomy the toric IOL 200, or portion thereof, shall be understood as referring to a representation of that item of anatomy, the toric IOL 200, or portion thereof in the image unless otherwise noted. The image received at step 402 may include the eye 100 of the patient and the toric IOL 200. An incision 500 through which the crystalline lens 112 was removed and the toric lens IOL was inserted may also be present.

The method 400 may include obtaining, at step 404, a label of the toric IOL 200 by processing the input image using the autoencoder 304. As shown in FIG. 5B, the label may include one or more pixel masks including labeled (e.g., non-zero) pixels at pixel positions corresponding to features of the toric IOL 200. For example, the label may include labeled pixels 502 for the marks 212, labeled pixels 504 for the perimeter of the peripheral ring 204, labeled pixels 506 for one or more bases 208 of the haptics 206. Labeled pixels may also be present for the rings 214, the incision, 500, or any items of anatomy of the eye 100 visible in the input image.

The method 400 may include obtaining, at step 406, an orientation of the toric IOL 200 from the label, or both the label and the input image using the autoencoder 304 as described above. The orientation may be in the form of line parameters (e.g., slope and x or y intercept) describing the IOL axis of the toric IOL 200.

The method 400 may include obtaining, at step 408, the reference axis for the treatment plan. For example, the reference axis may be received from the imaging device 302 or may be retrieved from a memory device or storage device in the computing device performing the method 400. The reference axis may be a result of a transformation performing during a registration step as described above. The reference axis obtained at step 408 may be in the form of line parameters (e.g., slope and x or y intercept) describing the reference axis relative to the input image. The reference axis may be obtained by performing a transformation of a reference axis defined with respect to a reference image in a treatment plan as described above.

The method 400 may include generating, at step 410, an output image including the input image having superimposed thereon representations of some or all of the label, reference axis, IOL axis, the direction indicator, and a representation of the difference in angle between the reference axis and IOL axis. The output image may then be displayed at step 412 on the display device 308.

For example, as shown in FIG. 5C, the output image generated at step 410 and displayed at step 412 may include some or all of a line 508 indicating the reference axis of the imaging device 302, a line 510 representing the IOL axis, a direction indicator 512 indicating a direction to rotate the toric IOL 200 to align the IOL axis with the reference axis, i.e., in the direction of the smallest subtended angle between the IOL axis and the reference axis. Some toric IOL 200 are safely rotatable in only one direction due to the shape of the haptics 206. Accordingly, the direction indicator 512 will always point in that direction, e.g. clockwise for the illustrated toric IOL 200. The output image may further include an indicator 514 in the form of digits, text or other symbolic representation (e.g., colors as described above) communicating one or both of the amount of rotation required (“X1 degrees”) and a direction of rotation required (clockwise (CW) or counter-clockwise (CCW)). In some implementations, the output image may include digits, text, or other symbolic representation 516 of a refractive error. The amount by which a misalignment between the reference axis and the IOL axis will affect the vision of the patient is proportional to the degree of astigmatism of the patient. The representation 516 of refractive error may therefore be computed as a function of the degree of astigmatism and difference in angle and superimposed on the output image in order to inform a surgeon when the toric IOL 200 is sufficiently aligned.

As shown in FIG. 4A the method 400 may be repeated. The method 400 may be repeated periodically for every frame of video feed from the imaging device 302 or may be repeated periodically at larger intervals, e.g., every N frames, where N is a value of 2 or more. The interval between frames for which the method 400 is performed may be from 1 ms to 2 seconds. The interval may depend on the processing power available to perform the method 400 and may be a smaller or larger than the values indicated herein.

For example, as shown in FIG. 5D, following display of the output image shown in FIG. 5C, a surgeon using a surgical instrument 518, e.g., an IOL fixation hook or other instrument, may rotate the toric IOL 200 as instructed. The method 400 may be repeated, resulting in an updated output image shown in FIG. 5D. As is apparent, the IOL axis (represented by line 510) is now more closely aligned with the reference axis (represented by line 508).

FIG. 5D further illustrates a scenario that may occur during alignment. Note that the instrument 518 in FIG. 5D is obscuring the marks 212 on one side of the peripheral ring 204 (see FIG. 5A). In other scenarios, marks 212 or the base 208 of a haptic may be positioned under the iris 106 and not be visible in the input image.

Such scenarios may be handled in various ways described below with reference to FIG. 4B. The process of generating the output image at step 410 may include receiving input data, where receiving the input data may include some or all of the receiving of the input image, at step 414, the receiving of the label output by the autoencoder 304, at step 416, and the receiving of a predicted label as output by the tracker 310, at step 418. In some implementations where tracker 310 is used, only the label as output by the tracker 310 is received at step 418. As noted above, the label from the tracker 310 may predict the location of some features, such as the marks 212 obscured by the instrument 518, the patient's iris 106, or due to some other cause.

The input data may then be processed, at step 420, such as using the line generator 312, which outputs, at step 422, line parameters describing the IOL axis. As described above, the line generator 312 may be embodied as a logistic regression model or other machine learning model. Since the label (either with or without predictions from the tracker 310) may include more than sufficient information to define a line, the line generator 312 may use unobscured features represented in the label to estimate the toric IOL axis with greater accuracy than a human. For example, one set of marks 212 on only one side of the peripheral ring 204 may be sufficient alone or in combination with the base 208 of only one of the haptics 206.

FIG. 5E illustrates an output image that may be displayed according to the method 400 following an iteration of the method 400 in which the difference in angle between the IOL axis and the reference axis is within a predefined tolerance. For example, the predefined tolerance may be a predefined angle difference such as an angle from 1 to 0.1 degrees. The predefined tolerance may be defined with respect to a refractive error: a tolerance is met when the refractive error for the difference in angle and the degree of astigmatism of the eye 100 is below a predefined error threshold.

Where the difference in angle meets the predefined tolerance, the output image may include an indicator 520 communicating that no further rotation is needed (NRR=no rotation required). The output image may include or omit labeled pixels 502, 504, 506, line 508, and line 510 when the difference in angle meets the predefined tolerance.

As shown in FIG. 5F, when the difference in angle meets the predefined tolerance, the surgical instrument 518 may then be withdrawn and any post-operative procedures may be performed as known in the art.

The foregoing description is provided to enable any person skilled in the art to practice the various embodiments described herein. Various modifications to these embodiments will be readily apparent to those skilled in the art, and the generic principles defined herein may be applied to other embodiments. Thus, the claims are not intended to be limited to the embodiments shown herein, but are to be accorded the full scope consistent with the language of the claims.

What is claimed is:

1. A method for providing alignment guidance during ocular surgery comprising:
(a) receiving, by a computing device, from an imaging device, an input image of a patient's eye having a toric intraocular lens (IOL) within the patient's eye;
(b) obtaining, by the computing device, a reference axis for the patient's eye, the reference axis indicating a desired orientation of a toric IOL axis of the toric IOL;
(c) processing, by the computing device, the input image to obtain a feature label indicating locations of features of the toric IOL represented in the input image, the features including any of: alignment dots defined on the toric IOL, a perimeter of the toric IOL, and portions of haptics of the toric IOL;
(d) processing, by the computing device, the feature label to determine an orientation of the toric IOL axis;
(e) calculating, by the computing device, an angle difference between the toric IOL axis and the reference axis;
(f) generating, by the computing device, an output image including at least one indicator corresponding to the angle difference; and
(g) outputting the output image to a display device.

2. The method of claim 1, further comprising:
(h) adjusting, by a surgeon, an orientation of the toric IOL; and
(i) repeating (a) through (g).

3. The method of claim 2, further comprising, following performing (h) and (i):
determining, by the computing device, that the angle difference meets a predefined tolerance; and
in response to determining that the angle difference meets the predefined tolerance, outputting, by the computing device, on the display device, an indicator indicating that no further rotation of the toric IOL is required.

4. The method of claim 3, wherein determining that the angle difference meets the predefined tolerance comprises determining that a refractive error resulting from the angle difference meets the predefined tolerance.

5. The method of claim 4, wherein (c) further comprises processing the input image to obtain one or more bounding boxes including the features and using the one or more bounding boxes to obtaining the feature label.

6. The method of claim 1, wherein processing the feature label to determine the orientation of the toric IOL axis comprises generating line parameters describing a line passing through the alignment dots.

7. The method of claim 6, wherein processing the feature label to determine the orientation of the toric IOL axis further comprises processing the feature label using a machine learning model.

8. The method of claim 7, wherein the machine learning model is a logistic regression model.

9. The method of claim 1, wherein the at least one indicator is one or more digits representing the angle difference.

10. The method of claim 1, wherein the at least one indicator is one or more digits representing a refractive error corresponding to the angle difference.

11. The method of claim 1, further comprising:
receiving, by the computing device, from the imaging device, a video feed comprising a plurality of frames;
performing (a) through (c) using each frame of the plurality of frames as the input image; and
tracking, by the computing device, using a tracking algorithm, the features for the plurality of frames to obtain a predicted label for each frame of the plurality of frames, the predicted label for one or more frames of the plurality of frames including representations of one or more of the features that are not represented in the feature label obtained for the one or more frames of the plurality of frames.

12. The method of claim 1, wherein the imaging device is a digital microscope.

13. The method of claim 1, further comprising:
matching, by the computing device, ocular anatomy represented in the input image to a treatment plan to determine an orientation of the patient's eye; and
determining, by the computing device, an orientation of the reference axis according to the treatment plan and the orientation of the patient's eye.

14. A system for providing alignment guidance during ocular surgery, the system comprising:
an imaging device;
a display device;
a computing device comprising one or more processing devices and one or more memory devices storing executable code that, when executed by the one or more processing devices, further cause the one or more processing devices to:
(a) receive from the imaging device, an input image of a patient's eye having a toric intraocular lens (IOL) within the patient's eye;
(b) obtain a reference axis for the patient's eye, the reference axis indicating a desired orientation of a toric IOL axis of the toric IOL;
(c) process the input image using a machine learning model to obtain a feature label indicating locations of features of the toric IOL represented in the input image, the features including any of: alignment dots defined on the toric IOL, a perimeter of the toric IOL, and portions of haptics of the toric IOL;
(d) process the feature label to determine an orientation of the toric IOL axis;
(e) calculate an angle difference between the toric IOL axis and the reference axis;
(f) generate an output image including at least one indicator corresponding to the angle difference; and
(g) output the output image to the display device.

15. The system of claim 14, wherein the executable code, when executed by the one or more processing devices, further causes the one or more processing devices to:
receive a video feed from the imaging device, the video feed comprising a plurality of frames; and
repeat (a) through (g) periodically using each frame of at least a portion of the plurality of frames as the input image.

16. The system of claim 15, wherein the executable code, when executed by the one or more processing devices, further causes the one or more processing devices to:
track, using a tracking algorithm, the features for the plurality of frames to obtain a predicted label for each frame of the plurality of frames, the predicted label for one or more frames of the plurality of frames including representations of one or more of the features that are not represented in the feature label obtained for the one or more frames of the plurality of frames.

17. The system of claim 15, wherein the executable code, when executed by the one or more processing devices, further causes the one or more processing devices to:
determine that the angle difference meets a predefined tolerance; and
in response to determining that the angle difference meets the predefined tolerance, output, on the display device, an indicator indicating that no further rotation of the toric IOL is required.

18. The system of claim 14, wherein the machine learning model is further configured to identify one or more bounding boxes highlighting the features and use the one or more bounding boxes to obtain the feature label.

19. The system of claim 14, wherein:
the machine learning model is a first machine learning model; and
the executable code, when executed by the one or more processing devices, further causes the one or more processing devices to process the feature label using a second machine learning model to determine an orientation of the toric IOL axis.

20. The system of claim 19, wherein the second machine learning model is a logistic regression model.
